(19) United States
(12) Patent Application Publication
(10) Pub. No.: US 2026/0047919 A1
(43) Pub. Date: Feb. 19, 2026

(54) BIOGLUE APPLICATIONS IN THE AORTA

(71) Applicant: Medtronic Vascular, Inc., Santa Rosa, CA (US)

(72) Inventors: Keith PERKINS, Santa Rosa, CA (US); Ian BARANOWSKI, Santa Rosa, CA (US); Zachary BORGLIN, Santa Rosa, CA (US); Tessa BRONEZ, Santa Rosa, CA (US)

(21) Appl. No.: 19/371,184

(22) Filed: Oct. 28, 2025

Related U.S. Application Data
(63) Continuation of application No. 17/749,591, filed on May 20, 2022.
(60) Provisional application No. 63/226,376, filed on Jul. 28, 2021.

(51) Int. Cl. A61F 2/07

(52) U.S. Cl. CPC A61F 2/07; A61F 2002/077

(57) ABSTRACT

A method including navigating a primary stent graft to a landing zone of a vessel, expanding the primary stent graft, navigating an adhesive catheter within the primary stent graft, deploying a secondary stent graft within the primary stent graft such that the adhesive catheter is trapped between the primary and secondary stent grafts, dispensing an adhesive from the adhesive catheter, and bonding the primary stent graft to the secondary stent graft with the adhesive.

### CROSS-REFERENCE TO RELATED APPLICATIONS

This application is a continuation of U.S. Application No. 17/749,591 filed on May 20, 2022, which claims the benefit of U.S. Provisional Application No. 63/226,376 filed on July 28, 2021, the disclosures of which are hereby incorporated by reference.

### TECHNICAL FIELD

The present technology is generally related to devices, systems, and methods for application of bioglue in blood vessels, such as the aorta.

### BACKGROUND

The use of endovascular procedures has been established as a minimally invasive technique to deliver a variety of clinical treatments in a patient's vasculature. A stent graft is an implantable device made of a tube-shaped surgical graft covering and an expanding or self expanding frame. The stent graft is placed inside a blood vessel to bridge, for example, an aneurismal, dissected, or other diseased or torn segment of the blood vessel, and, thereby, exclude the hemodynamic pressures of blood flow from the diseased segment of the blood vessel.

### SUMMARY

In a first embodiment, a method is disclosed. The method includes navigating a primary stent graft to a landing zone of a vessel, expanding the primary stent graft, navigating an adhesive catheter within the primary stent graft, deploying a secondary stent graft within the primary stent graft such that the adhesive catheter is trapped between the primary and secondary stent grafts, dispensing an adhesive from the adhesive catheter, and bonding the primary stent graft to the secondary stent graft with the adhesive.

In a second embodiment, a method is disclosed. The method includes navigating a primary stent graft to a landing zone of a vessel, expanding the primary stent graft, navigating an adhesive catheter within the primary stent graft and positioned radially within it, deploying a secondary stent graft within the primary stent graft, dispensing an adhesive from the adhesive catheter, and bonding the primary stent graft to the secondary stent graft with the adhesive.

In a third embodiment, a method is disclosed. The method includes navigating a primary stent graft to a landing zone of a vessel, expanding the primary stent graft, navigating an adhesive catheter within the primary stent graft, partially deploying a secondary stent graft within the primary stent graft, dispensing an adhesive from the adhesive catheter while the secondary stent graft is in its partially deployed state, and bonding the primary stent graft to the secondary stent graft with the adhesive.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIGS. 1A, 1B, 1C, and FIG. 1D are schematic illustrations of an adhesive delivery system used to treat aortic dissections, according to certain embodiments;

FIGS. 2A, 2B, and FIG. 2C are schematic illustrations of an adhesive delivery system and stents or stent grafts used to treat aortic dissections, according to certain embodiments;

FIG. 3 is a schematic illustration of an adhesive delivery system and an additional material to treat aortic dissections, according to certain embodiments;

FIG. 4 is a schematic illustration of an adhesive delivery system and stent graft used to treat an aortic aneurysm, according to certain embodiments;

FIGS. 5A, 5B, 5C, and FIG. 5D are schematic illustrations of an adhesive delivery system and stent graft assembly used to treat an aortic aneurysm, according to certain embodiments;

FIGS. 6A, 6B, and FIG. 6C are schematic illustrations of an adhesive delivery system used to treat an aortic aneurysm, according to certain embodiments;

FIGS. 7A, 7B, 7C, and FIG. 7D are schematic illustrations of an adhesive delivery system and stent graft assembly used to create a landing zone, according to certain embodiments;

FIG. 8 is a schematic illustrations of an adhesive delivery system used to treat a wall of an aortic aneurysm, according to certain embodiments;

FIGS. 9A and 9B are schematic illustrations of an inflatable adhesive delivery system and stent graft assembly, according to certain embodiments;

FIGS. 10A and 10B are schematic illustrations of an inflatable adhesive delivery system and stent graft assembly, according to certain embodiments;

FIGS. 11A, 11B, 11C, 11D, and FIG. 11E are schematic illustrations of a deployment sequence for an adhesive delivery system and stent graft assembly, according to certain embodiments;

FIGS. 12A and 12B are schematic illustrations of an adhesive delivery system using folded over graft material, according to certain embodiments;

FIGS. 13A and 13B are schematic illustrations of an adhesive delivery system with a groove, according to certain embodiments;

FIGS. 14A, 14B, 14C, and FIG. 14D are schematic illustrations of various fill lumens for an adhesive delivery system, according to certain embodiments;

FIGS. 15A, 15B, and FIG. 15C are schematic illustrations of various balloons for use with an adhesive delivery system, according to certain embodiments;

FIGS. 16A, 16B, 16C, 16D, 16E, 16F, 16G, and FIG. 16H are schematic illustrations of a process for making a perforated balloon material, according to certain embodiments;

FIGS. 17A, 17B, 17C, and FIG. 17D are schematic illustrations of a removable adhesive delivery system, according to certain embodiments; and

FIGS. 18A, 18B, and FIG. 18C are schematic illustrations of stent grafts with incorporated adhesives, according to certain embodiments.

### DETAILED DESCRIPTION

Embodiments of the present disclosure are described herein. It is to be understood, however, that the disclosed embodiments are merely examples and other embodiments can take various and alternative forms. The figures are not necessarily to scale; some features could be exaggerated or minimized to show details of particular components. Therefore, specific structural and functional details disclosed herein are not to be interpreted as limiting, but merely as a representative basis for teaching one skilled in the art to variously employ the embodiments. As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Directional terms used herein are made with reference to the views and orientations shown in the exemplary figures. A central axis is shown in the figures and described below. Terms such as "outer" and "inner" are relative to the central axis. For example, an "outer" surface means that the surfaces faces away from the central axis, or is outboard of another "inner" surface. Terms such as "radial," "diameter," "circumference," etc. also are relative to the central axis. The terms "front," "rear," "upper" and "lower" designate directions in the drawings to which reference is made.

Unless otherwise indicated, for the delivery system the terms "distal" and "proximal" are used in the following description with respect to a position or direction relative to a treating clinician. "Distal" and "distally" are positions distant from or in a direction away from the clinician, and "proximal" and "proximally" are positions near or in a direction toward the clinician. For the stent-graft prosthesis, "proximal" is the portion nearer the heart by way of blood flow path while "distal" is the portion of the stent-graft further from the heart by way of blood flow path.

The following detailed description is merely exemplary in nature and is not intended to limit the invention or the application and uses of the invention. Although the description is in the context of treatment of blood vessels such as the aorta, the invention may also be used in any other arteries or body passageways where it is deemed useful.

While endovascular stents and stent grafts effectively treat many types of aneurysms and dissections in the aorta and other blood vessels, there are certain disease states that remain challenging to address. For example, while a stent or stent graft may adequately seal the entry tear of a dissection, the false lumen created by the dissection may not be completely eliminated along the length of the dissection. Similarly, while stent grafts perform well at excluding abdominal aneurysms that have healthy tissue above the aneurysm and below the renal arteries (e.g., a landing zone), they may not be able to form an effective seal in hostile necks where there is little healthy tissue with which to form a seal. Accordingly, in embodiments described herein, biocompatible adhesives may be used on their own or as a supplement to stents or stent grafts to treat diseases of the aorta, such as aneurysms and dissections. Biocompatible adhesives may be referred to as simply an "adhesive" or a "bioglue." While the embodiments are described with reference to the aorta, the devices, systems, and methods are equally applicable to other blood vessels.

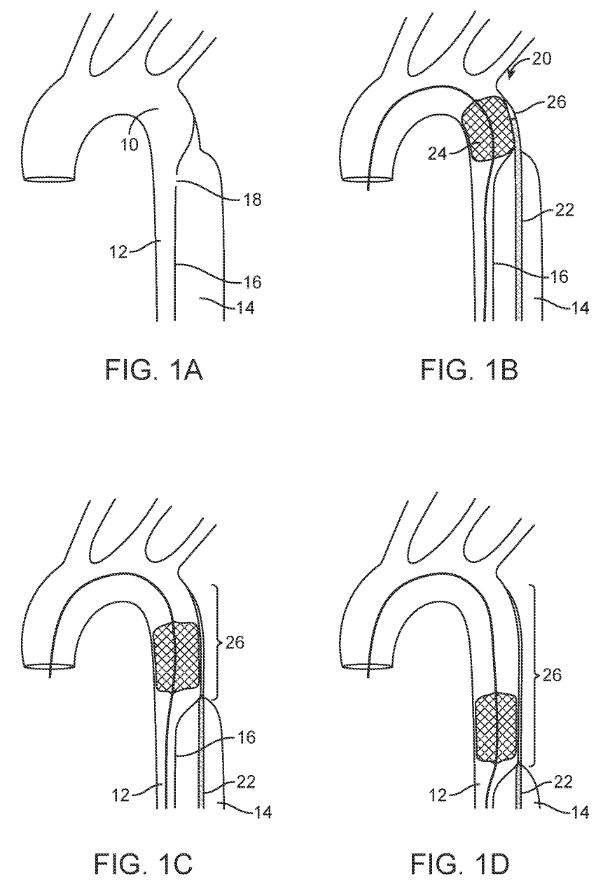

With reference to FIGS. 1A-1D, embodiments are disclosed for using an adhesive to reapproximate dissected tissue. FIG. 1A shows an example of a dissection of the descending aorta. While a dissection of the descending aorta is shown as an example, the following devices, systems, and methods may be used to treat dissections occurring in other portions of the aorta or in other vessels. The aorta 10 has a true lumen 12 and a false lumen 14 separated by a septum 16.

Blood may flow from the true lumen 12 to the false lumen 14 through an entry tear 18 (e.g., the primary entry tear). Dissections are dangerous, as blood flow into the false lumen reduces blood flow to downstream tissues and continued blood flow to the false lumen may cause the dissection to grow.

With reference to FIG. 1B, a bioglue system 20 is shown for reapproximating the dissected tissue to repair the dissection. The system 20 may include an adhesive delivery catheter 22 and a balloon catheter 24. As shown in FIG. 1B, the adhesive catheter 22 may be navigated to a proximal end of the false lumen 14. This may be done through the entry tear 18, through the false lumen 14 through a new opening created in the septum 16 (e.g., near a distal end of the false lumen 14, created by cutting, ablation, laser, etc.), or through an exit tear of the false lumen (not shown). The balloon catheter 24 may be navigated through the true lumen 12 to a position adjacent to the adhesive catheter 22 at the proximal end of the false lumen 14. The balloon catheter 24 may be any suitable balloon catheter known in the art, which may generally include a balloon that is inflatable via a fluid lumen and a fluid source (e.g., saline or air). The adhesive 26 may be delivered into the proximal end of the false lumen 14 via any suitable means, such as by a syringe or pump connected to the proximal end of the adhesive catheter 22. Once the adhesive 26 has been delivered, the balloon catheter 24 may be inflated to reapproximate the septum with the wall of the aorta and provide compression while the adhesive cures, sets, or polymerizes. In addition to applying pressure to the tissue and adhesive, the balloon may also reduce or stop the flow of blood in the aorta to provide a low flow environment for the adhesive to cure and reduce the risk of embolization of the adhesive.

With reference to FIGS. 1C and 1D, the delivery of the adhesive 26 may continue distally along the length of the dissection until the false lumen 14 is eliminated. In one embodiment, the adhesive catheter 22 may deliver a set amount of adhesive 26 and/or may be retracted a set distance and then the balloon catheter 24 may be inflated to reapproximate a section of the septum 16 generally corresponding to a length of the balloon. The adhesive catheter 22 may then deliver another amount or length of adhesive 26 to a section of the aorta distal to the reapproximated section and the process may be repeated along the length of the dissection from its proximal end to its distal end. In between reapproximation steps, the balloon may be deflated (partially or entirely) in order to allow blood flow through the true lumen 12 and to allow the balloon catheter to be retracted in the distal direction. In another embodiment, the balloon may remain inflated and may be pulled distally after each bolus of adhesive 26 is delivered.

In another embodiment, instead of delivering the adhesive 26 in discrete steps and performing an inflation step after each bolus of adhesive 26 is delivered, the adhesive catheter 22 may be continuously retracted while dispensing adhesive 26. The balloon catheter 24 may be retracted along with the adhesive catheter (e.g., slightly proximal or after) to reapproximate the septum 16 with the wall of the aorta 10 in a single, continuous step.

FIGS. 1A-D show a single adhesive catheter 22 creating a single line or strip of adhesive 26 down the false lumen 14. This single strip of adhesive 26 may be effective to close the false lumen 14 and the pressure from the balloon may cause the adhesive 26 to spread and create a larger contact area for the adhesive 26 between the septum 16 and the vessel wall. In one embodiment, the balloon may have grooves or channels therein, for example in the circumferential direction, which may cause the adhesive 26 to spread circumferentially when the balloon is inflated and applies pressure to the adhesive 26.

Nevertheless, in other embodiments, multiple adhesive catheters may be used to create multiple strips of adhesive. For example, multiple adhesive catheters may be used to create circumferentially spaced apart adhesive strips similar to the single strip shown in Figs. lB-1D. Alternatively, the adhesive catheter 22 may be moved circumferentially as it delivers adhesive, either prior to moving it distally or as it is moved distally. If it is moved circumferentially as it is moved distally, the adhesive may be delivered in a generally sinusoidal pattern. The adhesive catheter 22 may have a deflectable tip, such that it can move from a straight orientation to a bent orientation, in order to facilitate delivery over a wider area. In these embodiments, when the balloon is inflated it may cause adhesive to cover a greater area of the septum and vessel wall than a single strip done essentially in the longitudinal direction.

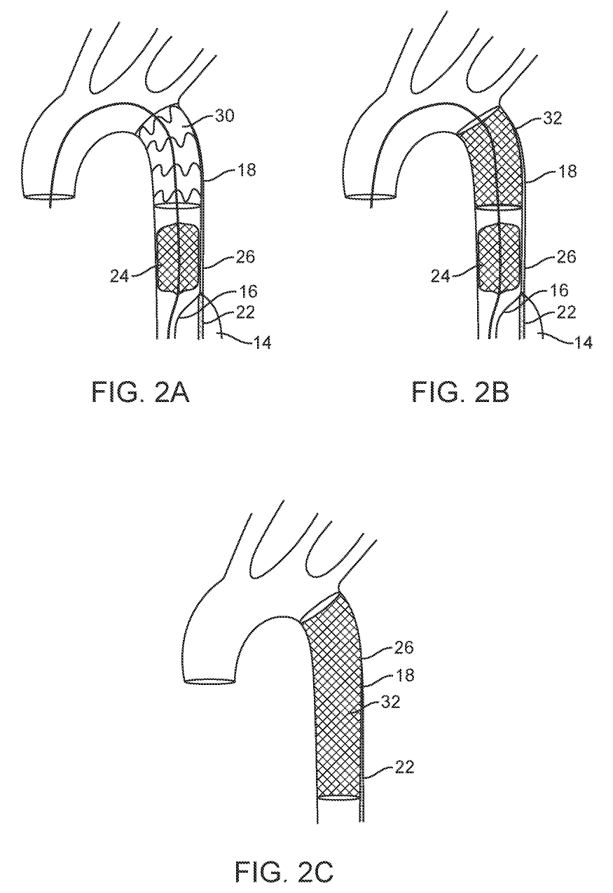

With reference to FIGS. 2A-2B, additional embodiments are shown for using the bioglue system 20 with an additional stent graft 30 or open cell stent 32, generally referred to as an endovascular device. In these embodiments, the majority of the process for reapproximating the septum 16 may be similar to the process shown and described with respect to FIGS. 1A-1D, but with the addition of either a stent graft 30 (FIG. 2A) or open cell stent 32 (FIG. 2B) to cover or close the entry tear 18. The stent graft 30 or open cell stent 32 may be positioned to overlap with the entry tear 18 but may also extend proximal to the entry tear 18 in order to form a seal with the aorta.

The stent graft 30 may be any suitable stent graft, generally include a tubular graft material and one or more stents attached thereto. The stent graft 30 may be self-expanding (e.g., using nitinol stents) or balloon expandable. The graft material may be impermeable or substantially impermeable to blood, such that upon deployment the entry tear 18 is covered and blood may not enter the false lumen 14. In embodiments using an open cell stent 32, the stent 32 may similarly be self-expanding or balloon expandable but without a graft material. Accordingly, openings between the stent wires or in the stent mesh may allow blood to pass therethrough. However, the open cell stent 32 may effectively reapproximate the septum 16 to the wall of the aorta 10, thereby closing off the false lumen 14 and preventing additional blood from flowing therein. The adhesive 26 may further facilitate the closing of the entry tear 18.

In the embodiments described with respect to FIGS. 2A and 2B, the adhesive 26 may be delivered into the false lumen 14 prior to expansion of the stent graft 30 or open cell stent 32. The stent graft 30 or open cell stent 32 may then provide the pressure to reapproximate the septum 16. Accordingly, in some instances, the balloon catheter 24 may not be used in portions of the aorta that are covered by the stent graft 30 or open cell stent 32. In other embodiments, the balloon catheter 24 may be deployed within the stent graft 30 or open cell stent 32 to provide pressure in addition to the radial force of the stent graft 30 or open cell stent 32. Distal to the distal end of the stent graft 30 or open cell stent 32, the process for reapproximating the septum may be similar to that described with respect to FIGS. 1A-1D.

With reference to FIG. 2C, an open cell stent 32 may be used to cover more than just the entry tear 18. In one embodiment, the open cell stent 32 may have a length that is equal to or greater than a length of the dissection. The open cell stent 32 may therefore provide a reapproximation force along the entire length of the dissection. In one embodiment, the adhesive catheter 22 may deliver adhesive 26 along the length of the dissection prior to expansion of the open cell stent 32. In another embodiment, the open cell stent 32 may be expanded with the adhesive catheter 22 still within the false lumen 14. The adhesive catheter 22 may then be sequentially or continuously retracted as it delivers adhesive 26 and the open cell stent 32 may expand further as the adhesive catheter 22 moves. In at least one embodiment, the approach of FIG. 2C does not include the use of a balloon to apply a reapproximation force. In other embodiments, a balloon catheter 24 may be used from within the open cell stent 32, as described with reference to FIGS. 2A and 2B.

In one embodiment, the open cell stent 32 can be covered or paved with a bioabsorbable scaffolding material that temporarily prevents or significantly reduces blood flow through the open cells of the stent 32. This may prevent blood flow through the open cell stent 32 from entering the false lumen 14 through small tears in the septum 16 other than the entry tear 18. This blood flow may otherwise displace the adhesive in the false lumen 14 during delivery and reduce its effectiveness or cause an embolic risk. By providing a bioabsorbable scaffolding or membrane over the open cell stent 32, an acute favorable low flow environment may be created for glue application, but the scaffolding will dissolve or be absorbed after a tunable period of time. Therefore, in the long-term, only an open cell stent remains, which may be free to be incorporated into the vessel wall.

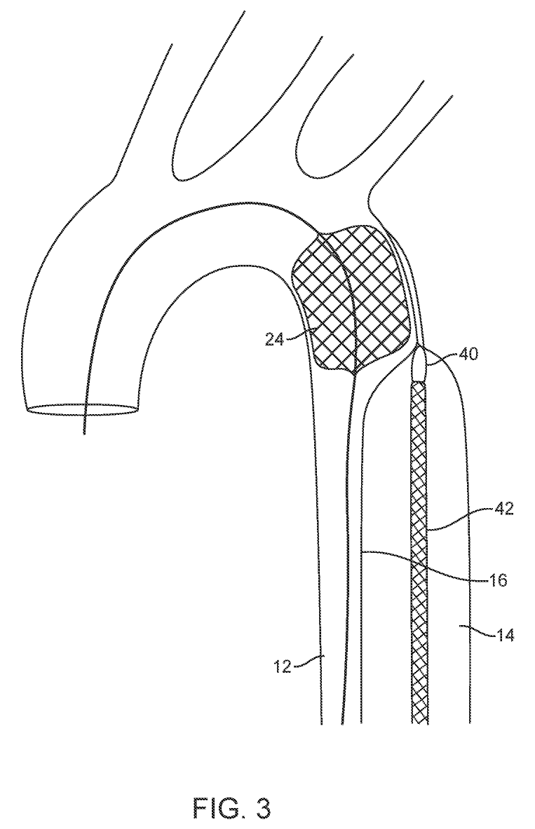

With reference to FIG. 3, an embodiment of the delivery of an adhesive-infused material 40 is shown. This embodiment may be similar to that shown and described with reference to FIGS. 1A-1D, but instead of delivering an adhesive 26, an adhesive-infused material 40 is delivered into the false lumen 14. In one embodiment, the adhesive-infused material 40 is an adhesive-infused coil or an adhesive-infused surgical material (e.g., mesh, felt, shape memory material (metal or polymeric)) having a high surface area to stabilize the interaction of the adhesive and the vessel tissue (e.g., septum). The adhesive-infused material 40 may be delivered using a delivery catheter 42.

The delivery method for the embodiments of FIG. 3 may be similar to that of FIGS. 1A-1D. The delivery system 42 may be either sequentially or continuously retracted and the balloon catheter 24 may apply a reapproximation force. The adhesive-infused material 40 may provide additional support to the fragile tissue of the septum 16 compared to an adhesive alone. The adhesive-infused material 40 also provides an increased surface area inside the false lumen 14 for platelet activation and blood clot formation. The adhesive-infused material 40 may promote stasis within the false lumen 14 and promote false lumen thrombosis. Similar to the embodiments of FIGS. 2A-2C, stent grafts or open cell stents may be used in conjunction with embodiments having the adhesive-infused material 40.

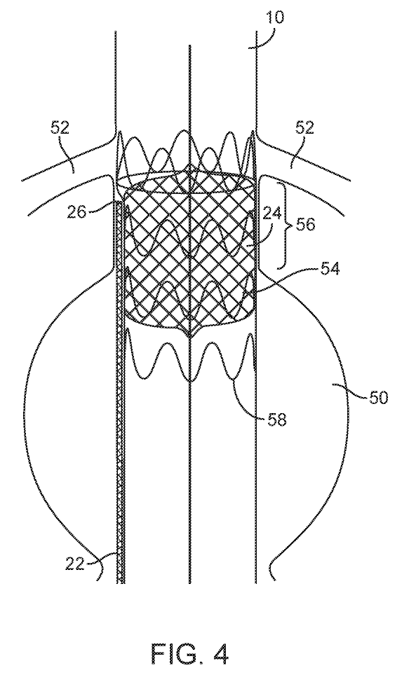

With reference to FIG. 4, embodiments are disclosed wherein the adhesive 26 may be used to improve migration resistance and prevention of type I endoleaks (a gap between the graft and the vessel wall) for stent grafts. FIG. 4 shows an abdominal region of the aorta 10 with an abdominal aneurysm 50 located just distal to the renal arteries 52. Such aneurysms may provide a relatively short landing zone 56 for the stent graft 54 to form a seal with the vessel wall. A short landing zone 56 may have fewer stent rings 58 located therein, which may reduce the migration resistance and/or sealing capability of the stent graft 54.

In at least one embodiment, an adhesive catheter 22 may be used to deliver adhesive 26 between the stent graft 54 and the vessel wall in the landing zone 56. The adhesive catheter 22 may be positioned between the vessel wall and the stent graft 54 prior to expanding the stent graft 54. The adhesive 26 may be delivered (e.g., on the vessel wall) and the stent graft 54 may then be expanded to contact the adhesive 26 and the vessel wall, thereby applying radial force and creating a good bond between the vessel and the stent graft 54 via the adhesive 26. Alternatively, the stent graft 54 may be expanded while the adhesive catheter 22 is in the delivery position but prior to dispensing the adhesive 26, thereby trapping the adhesive catheter 22 between the vessel wall and the stent graft 54. The adhesive 26 may then be delivered between the vessel wall and the expanded stent graft 54.

The adhesive 26 may be delivered at or near the proximal end of the stent graft 54 to improve the seal of the stent graft and reduce type I endoloeaks. In addition or alternatively, the adhesive 26 may be delivered distal to the proximal end to improve migration resistance. Similar to above embodiments, the adhesive catheter 22 may be sequentially continuous retracted to deliver the adhesive 26 along a length of the stent graft 54. Devices and systems are described below to distribute the adhesive 26 around a circumference of the stent graft 54, which may be used with any of the embodiments described in the present application. Alternatively, multiple adhesive catheters 22 may be introduced between the vessel wall and the stent graft 54, for example, circumferentially spaced about the stent graft 54, to deliver the adhesive 26 at multiple locations. As described in earlier embodiments, a balloon catheter 24 may be used to apply additional outward pressure on the graft material during the adhesive curing/polymerization and/or to block or slow the blood flow in the aorta 10 to reduce the risk of embolization. The balloon may have circumferential grooves or channels, which may cause the adhesive 26 to spread circumferentially when pressure is applied. In addition, the fabric of the stent may wick or guide the glue circumferentially such that the bond is spread over a larger area than where the adhesive catheter 22 delivers the adhesive 26.

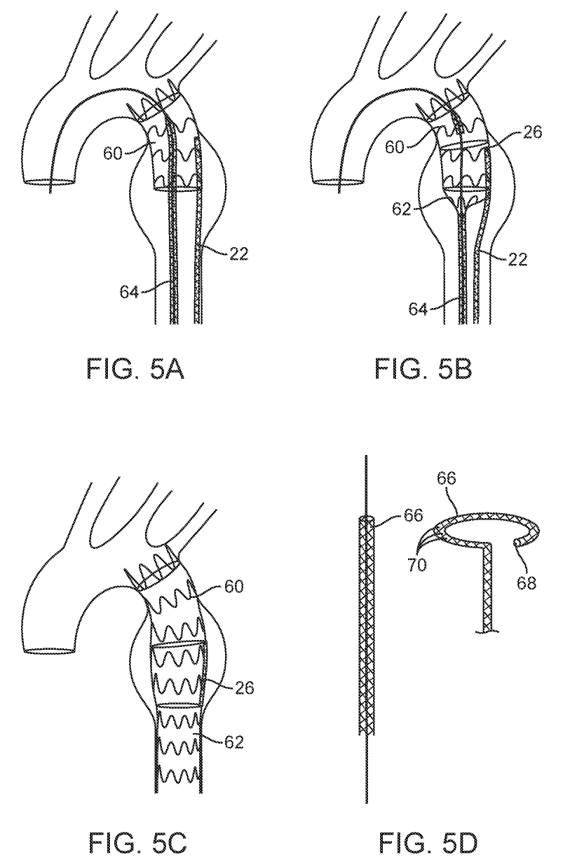

With reference to FIGS. 5A-5D, embodiments are shown wherein the adhesive 26 may be used to lock together and prevent separation or migration of modular stent graft components. As shown in FIG. 5A, a first or primary stent graft 60 may be deployed in the aorta 10 (the descending aorta, as shown). The adhesive catheter 22 may be navigated to the primary stent graft 60, positioned radially within it.

With reference to FIG. 5B, a secondary stent graft 62 may be configured to be deployed within the primary stent graft 60 in order to create a modular stent assembly. The secondary stent graft may be deployed using a delivery system 64. As shown in FIG. 5B, the secondary stent graft 62 may be partially deployed within the primary stent graft 60 such that adhesive catheter 22 is trapped or sandwiched between the two stent grafts 60, 62. The delivery system 64 may keep the secondary stent graft 62 in a partially expanded state, with at least a portion of the stent graft 62 still compressed within the delivery system 64. The adhesive catheter 22 may then deliver the adhesive 26 between the two stent grafts 60, 62. Similar to above embodiments, a balloon catheter 24 may be used to inflate a balloon in the region where the two stent grafts 60, 62 overlap and the adhesive 26 has been delivered, in order to force the stent grafts 60, 62 together and/or to reduce blood flow.

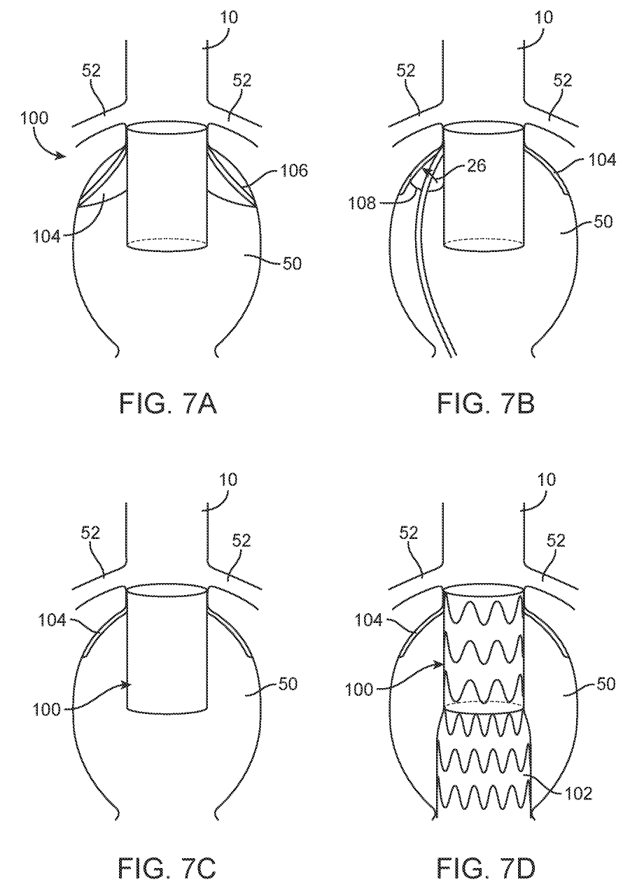

With reference to FIG. 5C, once the adhesive 26 has been delivered, the adhesive catheter 22 may be removed and the delivery system 64 may be used to complete the deployment of the secondary stent graft 62. The adhesive 26 may be delivered over at least a portion of the overlapped region of the two stent grafts 60, 62. In one embodiment, the adhesive 26 may be delivered to substantially the entire length of the overlapped region. As described with respect to FIG. 4, multiple adhesive catheters 22 may be used or the adhesive catheter 22 may be configured to distribute the adhesive 26 around the circumference of the stent grafts 60, 62. With reference to FIGS. 7A-7D, embodiments are shown for creating an engineered seal zone using a bioglue. With reference to FIG. 7A, an abdominal aneurysm 50 is shown in the aorta 10. A docking graft 100 is shown deployed within the aneurysm 50 just distal to the renal arteries 52. The docking graft 100 may be similar to the stent grafts described elsewhere in the present disclosure, for example, including a graft material and optionally one or more stent rings. The docking graft 100 may have a tubular or cylindrical shape, which may provide a landing zone for securing or docking a second stent graft 102 (e.g., as shown in FIG. 7D).

With reference to FIG. 5D, one example of a tip 66 for adhesive catheter 22 is shown. The tip 66 may be shape set such that it has a relaxed or natural configuration and a temporary or stressed position. In the embodiment shown, the tip 66 may have an annular or ring-like shape in its relaxed or natural state (e.g., on the right). In order to facilitate navigating the adhesive catheter 22 to the delivery site, the tip 66 may be temporarily held in a straight configuration (e.g., on the left). The tip 66 may be shape set and switched between relaxed and stressed configurations in any suitable manner. In one example, a wire, such as a guide wire may be extended through the tip 66 to straighten it and hold it in the delivery configuration. Upon removal of the wire, the tip 66 may revert to its relaxed or shape set configuration. Other possibilities may include a sleeve that holds it straight as is removed or temperature-based shape setting.

The tip 66 may have a distal opening 68 at the end of the tip 66 for the delivery of the adhesive 26 and/or it may have one or more lateral openings 70 in the side wall to delivery adhesive 26. Accordingly, a single catheter and tip 66 may be used to release adhesive 26 around the circumference of the stent grafts. In one embodiment, the tip 66 may form substantially a complete circle such that an entire circumference of the stent graft may receive adhesive 26. In other embodiments, the tip 66 may form an arc that provides adhesive to a portion of the circumference. Multiple catheters may be used to apply adhesive to the entire circumference or a single catheter may be rotated to deliver adhesive to the entire circumference. In other embodiments, adhesive around only a portion of the circumference may be sufficient to provide adequate separation or migration resistance. Once the adhesive 26 has been delivered, the tip 66 may be reverted back to the delivery configuration (e.g., wire re- inserted) and the adhesive catheter 22 may be removed. If a ring-shaped tip is used, it may be beneficial to navigate the tip 66 into the primary stent graft 60 (e.g., at the distal end) and cause it to take its annular or ring shape prior to navigating the secondary stent graft 62 into position. Therefore, the secondary stent graft 62 may be deployed within the ring shaped tip 66 and adhesive 26 may be delivered.

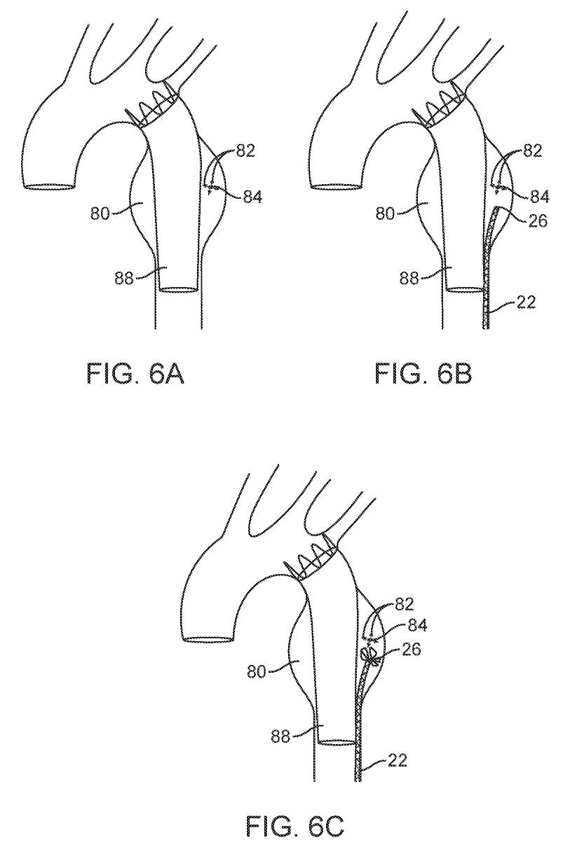

With reference to FIGS. 6A-6C, the adhesive 26 may be used to treat type II endoloeaks (collateral retrograde flow from branch vessels of the aorta). FIG. 6A shows an aneurysm 80 with a type II endoleak 82 therein. The ostia 84 of a collateral vessel is shown as a circle, with the arrows indicated blood flow entering the aneurysm 80 therethrough. A stent graft 88 has been deployed to exclude the aneurysm 80, but without further treatment the type II endoleak will continue to pressurize the aneurysm sac.

With reference to FIG. 6B, an adhesive catheter 22 is shown within the aneurysm 80. The adhesive catheter 22 may be tunneled between the stent graft 88 and the vessel wall after the stent graft 88 has been deployed. In one embodiment, a guidewire may be positioned in the aneurysm 80 prior to deploying the stent graft 88, such that the adhesive catheter 22 can be tracked over the guidewire to more easily pass between the stent graft 88 and the vessel wall. Alternatively, the adhesive catheter 22 may be in place prior to full expansion of the stent graft 88 such that it is trapped between the stent graft 88 and the vessel when the stent graft 88 is expanded. In other embodiments, the adhesive catheter 22 may be inserted via puncture of the aneurysm sac (e.g., translumbar) or by access through a collateral vessel (e.g., the collateral vessel to be sealed or others within the sac).

Once the adhesive catheter 22 is in the desired position at, near, or within the ostia 84, the adhesive 26 may be delivered. The adhesive 26 may be delivered within the ostia 84, deeper within the collateral vessel, or around/over the ostia 84 to effectively seal the ostia 84 and prevent blood flow therefrom. Alternatively, if there is thrombus or other material surrounding the ostia 84, but there is a flow path through the material from the ostia 84 to the sac, the adhesive 26 may be delivered to that flow path to effectively seal off the collateral vessel. Similar to FIG. 3, the adhesive 26 may be delivered in combination with coils or thrombus-generating devices to eliminate the endoleak. The adhesive 26 may help lock the three-dimensional shape of the coils or other devices in place.

With reference to FIGS. 7A-7D, embodiments are shown for creating an engineered seal zone using a bioglue. With reference to FIG. 7A, an abdominal aneurysm 50 is shown in the aorta 10. A docking graft 100 is shown deployed within the aneurysm 50 just distal to the renal arteries 52. The docking graft 100 may be similar to the stent grafts described elsewhere in the present disclosure, for example, including a graft material and optionally one or more stent rings. The docking graft may have a tubular or cylindrical shape, which may provide a landing zone for securing or docking a second stent graft 102 (e.g., as shown in FIG. 7D).

The docking graft 100 may have a sealing member or sealing skirt 104 to improve seal and/or fixation of the docking graft. The sealing skirt 104 may be particularly beneficial in situations where there is a small or complicated landing zone, such as a short neck, a wide neck, or tortious vessel anatomy. The sealing skirt 104 may attach to or extend from a proximal end of the docking graft 100 and may extend around the full circumference of the docking graft 100. Accordingly, once deployed, the sealing skirt 104 may have a frustoconical or tent-like shape, with a smaller diameter at the top/proximal end and a larger diameter at the bottom/distal end. While the sealing skirt 104 is shown here to provide a proximal seal zone, it may also be used to provide a distal seal zone, in which case the above description would be inverted.

In one embodiment, the sealing skirt 104 may include wires 106 that are configured to open the sealing skirt 104 and create apposition with the vessel wall. The wires 106 may be formed of a shape memory material, such as nitinol and may transition from a compressed configuration to an expanded configuration during delivery. The wires 106 may have any arrangement to cause the sealing skirt 104 to contact the vessel wall, such as circumferential rings or longitudinal lines.

The sealing skirt 104 may be secured to the vessel wall to anchor the docking graft 100 in place and provide a robust connection point for the second stent graft 102. In at least one embodiment, the sealing skirt 104 may be secured to the vessel wall by adhesive 26, which may be delivered using adhesive catheter 22. The adhesive 26 may be delivered between the sealing skirt 104 and the vessel wall or it may be applied to the inner surface of the sealing skirt 104 and may diffuse through the skirt material to bond with the vessel wall. In addition to or instead of adhesive, other mechanisms may be used to secure the sealing skirt 104 to the vessel wall. For example, endoanchors or endosutures may be used, such as HELI-FX endoanchors from MEDTRONIC. Other attachment mechanisms could include active fixation, such as hooks or barbs that are configured to embed in the vessel wall. In one embodiment, no adhesive or additional fasteners may be used, but rather the self-expanding wires 106 may provide sufficient apposition force to provide fixation and reduce migration.

With reference to FIG. 7B, in one embodiment, the adhesive 26 may be applied to the sealing skirt using a weeping balloon 108. The weeping balloon 108 may have holes or openings therein, such that when adhesive 26 is introduced into the balloon under pressure, some of the adhesive 26 is released through the openings and is transferred to the surface in contact with the balloon. As shown in FIG. 7B, the weeping balloon 108 may be used to apply pressure to the sealing skirt 104 to push it into the vessel wall while also releasing adhesive 26 through the openings. The pressure applied by the balloon may force the adhesive 26 into and through the skirt material so that the sealing skirt 104 is bonded to the vessel wall.

With reference to FIG. 7C, the docking graft 100 is shown secured in place after the adhesive or other attachment mechanisms have been deployed. The docking graft 100 may therefore form a robust connection point for second stent graft 102. The engineered seal zone may also stop the proximal progress of the disease by stabilizing the tissue. As described above, the docking graft 100 with sealing skirt 104 may be applied to any type of seal/landing zone, whether proximal or distal.

With reference to FIG. 7D, the docking graft 100 is shown with a second stent graft 102 secured thereto, with a proximal portion of the second stent graft 102 deployed within a distal portion of the docking graft 100. As shown herein, the docking graft 100 may include stent rings in some embodiments, but in others it may not (e.g., FIG. 7A).

With reference to FIG. 8, embodiments are shown of using a bioglue to stabilize the aneurysm sac. The adhesive 26 may be applied directly to the wall of the aneurysm 50 using an applicator 120, which may be similar to adhesive catheter 22 in some embodiments. The applicator 120 may include a lumen through which adhesive may be delivered to the aneurysm wall. The applicator 120 may apply a coating of the adhesive 26 on the inner surface of the aneurysm sac. For example, the adhesive 26 may be applied to a majority (e.g., at least 50%) of a surface area of the aneurysm sac. In other embodiments, the adhesive may coat at least 75%, 90%, or 95% of the surface area of the aneurysm 50. In one embodiment, the entire surface (e.g., 100%) of the aneurysm 50 may be coated. The applicator 120 may have a deflectable tip to facilitate application of the adhesive to the aneurysm wall. For example, it may include one or more wires extending in the longitudinal direction that may be tensioned to cause the tip to deflect in a desired direction. One example of such a deflectable catheter may be the HELI-FX GUIDE, from MEDTRONIC.

In another embodiment, a balloon may be used to deliver adhesive to the aneurysm wall instead of an applicator 120. A balloon catheter may be tracked to the aneurysm 50 and a balloon may be inflated such that an outer surface of the balloon contacts the aneurysm wall. The balloon may be conformable, such that it adapts its shape to the contours of the aneurysm 50. The balloon may be sized and configured to contact a certain surface area of the aneurysm 50 that is desired to be coated with adhesive, such as at least 50%, 75%, 90%, 95%, or substantially 100%. The exterior surface of the balloon may be coated with an adhesive such that the adhesive is transferred to the aneurysm wall upon contact therewith. In another embodiment, the balloon may have perforations therein and adhesive may be delivered from within the balloon and may diffuse or travel through the perforations to the exterior of the balloon and contact the aneurysm wall. Additional details of suitable balloons and their features are described with reference to FIGS. 7A-7D, 15A-15C, and FIGS. 16A-16H.

By coating the aneurysm in the adhesive 26, the properties of the aneurysm tissue may be changed and converted to a condition that is more resistant to rupture and/or disease progression. The adhesive itself may also resist aneurysm expansion and rupture. In at least one embodiment, the adhesive 26 is only applied as a coating or relatively thin layer on the aneurysm wall. In other words, the aneurysm sac is not filled with the adhesive. Delivery of the adhesive through the applicator 120 from an adhesive source may be via any suitable method, such as by syringe or pump. In another embodiment, the distal end of the applicator 120 may include a roller that may receive adhesive and then spread the adhesive onto the aneurysm wall, similar to a ballpoint pen. In another embodiment, the distal end of the applicator 120 may have an absorbent portion that may receive adhesive and transfer it to the aneurysm wall, similar to a felt-tip marker.

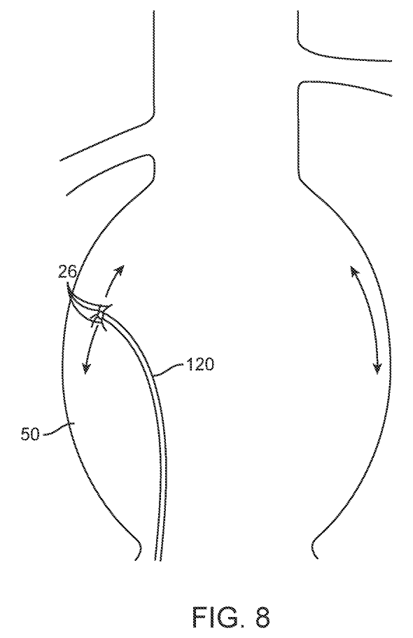

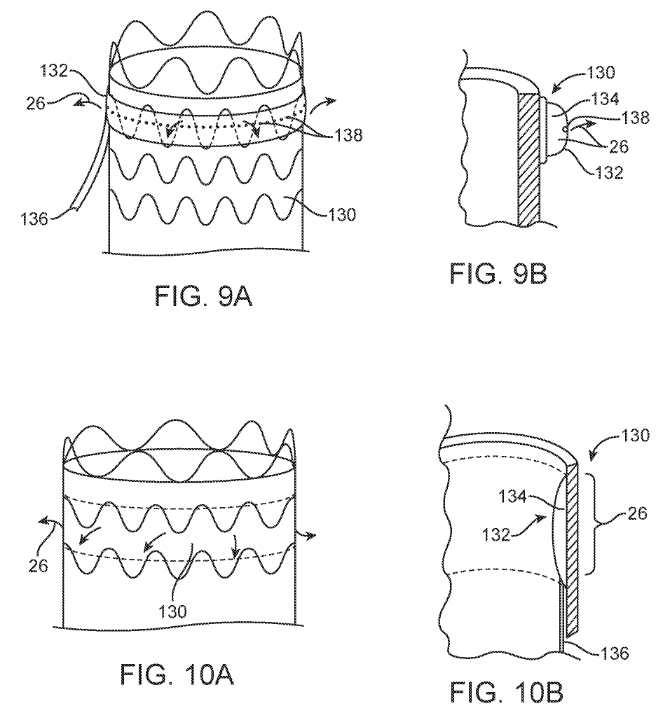

With reference to FIGS. 9A and 9B, embodiments of an inflatable adhesive infusion system are shown. FIG. 9A shows a perspective view and FIG. 9B shows a partial cross-section. A stent graft 130 is shown, which may be similar to the other stent grafts disclosed herein (e.g., tubular graft materials with one or more stent rings). A hollow member 132 may be disposed circumferentially about at least a portion of the stent graft 130, for example, around a proximal portion of the stent graft 130 that is configured to seal to a vessel wall or to another stent graft. The hollow member 132 may also be referred to as a bag or tube/tubing. In one embodiment, the hollow member 132 extends around an entire circumference of the stent graft 130. The hollow member 132 may have a cavity 134 therein, which may be configured to receive the adhesive 26 through a fill lumen 136. The hollow member 132 may have one or more (e.g., a plurality) of holes or pores 138 on its radially external surface.

The adhesive 26 may be delivered from an adhesive source (e.g., syringe) through the fill lumen 136 into the hollow member 132, where it fills the cavity 134. The adhesive may then flow through the holes 138 to contact the vessel wall and form a bond therewith. The radial force of the stent graft 130 (from either self-expansion and/or balloon expansion) may press the hollow member 132 against the vessel wall to facilitate good contact between the hollow member, the adhesive, and the vessel wall. Once the adhesive cures or sets, the stent graft 130 may be bonded to the vessel wall via the hollow member 132 and adhesive 26. In some embodiments, the holes 138 may also be on the radially interior side of the hollow member 132, such that the adhesive flows therethrough and is in direct contact with the graft material and/or stents of the stent graft 130. In addition to or alternatively to holes 138, the material of the hollow member 132 may be porous such that the adhesive 26 may seep through the hollow member 132 to contact the vessel wall and the graft material or stents of the stent graft 130. The hollow member 132 may have a complete, continuous outer wall enclosing the cavity 134 or the hollow member may be a sheet of material that is coupled on two sides to the stent graft 130 to form the cavity 134 between an outer surface of the stent graft and the hollow member 132.

With reference to FIGS. 10A and 10B, embodiments of another inflatable adhesive infusion system are shown. The system of FIGS. 10A, 10B is similar to that of FIGS. 9A, 9B, except that the hollow member 132 is on the inner circumference of the stent graft 130 instead of the outside. Since the inner surface of the hollow member 132 would be exposed to blood flow in this embodiment, at least the inner surface may be impermeable to the adhesive 26 (e.g., non-porous, no holes). The outer surface may have holes or pores 138, similar to above, or it may be porous to allow the adhesive to flow through to the graft material of the stent graft 130. Alternatively, as disclosed above, the hollow member 132 may be formed as a single sheet or layer of material that is attached to the stent graft so as to create the cavity 134 therebetween. In this embodiment, the adhesive 26 may seep from the cavity 134 through the stent graft material and contact that vessel wall. Such embodiments may reduce the profile of the stent graft 130 for either the inner or outer hollow member configuration (FIGS. 9A, 9B or FIGS. 10A, 10B). The embodiments of FIGS. 10A, 10B may have the benefit of an unchanged outer diameter of the stent graft 130. In addition, delivering the adhesive on the inside of the stent graft 130 may create outward pressure on the stent graft 130 and generate better apposition with the vessel wall.

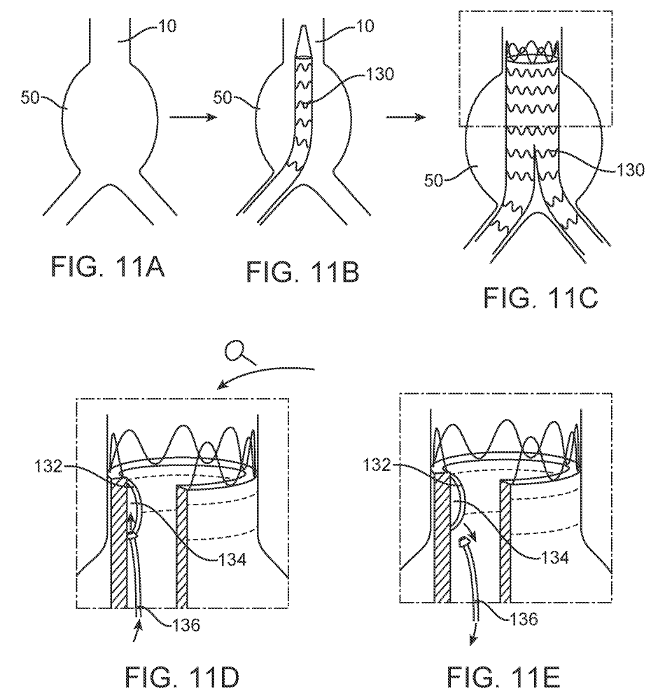

With reference to FIGS. 11A-11E, an example deployment sequence is shown for the delivery of a stent graft 130 having a hollow member 132. While the steps are shown with respect to an inner hollow member 132, such as shown in FIG. 10, the steps may also apply to an outer hollow member 132 (FIGS. 9A, 9B). FIG. 11A shows an aneurysm 50 in the aorta 10. FIG. 11B shows the stent graft 130 being tracked to the aneurysm in a compressed configuration via femoral access. FIG. 11C shows the stent graft 130 in a deployed position. The stent graft 130 may be tracked to and deployed in the aneurysm using any suitable delivery system and method.

As shown in FIGS. 11C and 11D, the fill lumen 136 may be pre-installed or connected to the hollow member 132 prior to stent graft deployment. In such embodiments, delivery of the adhesive may be performed once the stent graft 130 is deployed. In other embodiments, a fill lumen or fill catheter may be navigated to the stent graft 130 and inserted into the hollow member 132 after the stent graft 130 has been deployed. With reference to FIG. 11E, once the adhesive has been delivered to the cavity 134, the fill lumen 136 may be removed or detached from the hollow member 132 and retracted from the patient.

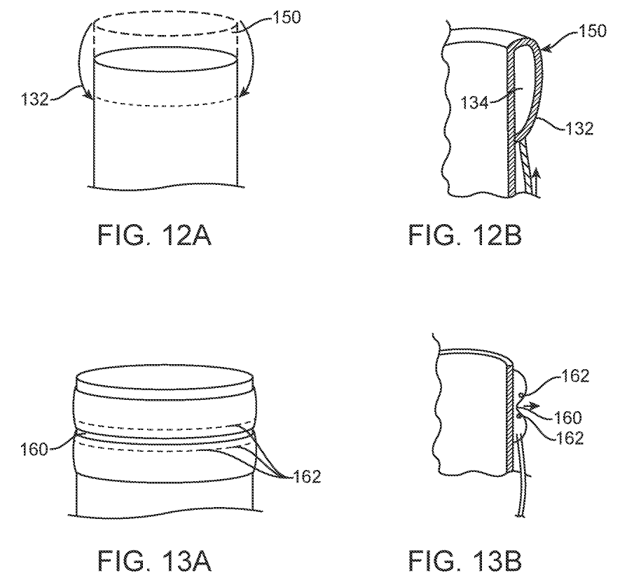

With reference to FIGS. 12A and 12B, embodiments are shown for alternatives to the hollow members 132 shown and described in FIGS. 9A, 9B and 10A, 10B. In these embodiments, rather than a separate hollow member 132, a portion of excess fabric 150 at the end of the stent graft (proximal end shown, but may be distal end) may be folded over to create the cavity 134. The excess fabric 150 may be sewn or otherwise attached to the graft material on either the outside of the stent graft (as shown) or the inside of the stent graft to create an external or internal cavity 134 similar to those shown and described in FIGS. 9A, 9B and 10A, 10B. The deployment process may be similar to that described in FIG. 11A-11E.

With reference to FIGS. 13A and 13B, embodiments are shown of a modification to the hollow member 132 or folded over fabric 150 from previous embodiments. In these embodiments, a recess or groove 160 may be formed in the hollow member 132 or the folder over fabric 150, whichever is used, to focus or direct the adhesive into a ring. The groove 160 may be formed in any suitable manner, such as by stitching or sutures, heat/shape setting, a constraining loop, or others. Holes or openings 162 may be located on and/or adjacent the groove 160 such that adhesive that flows therethrough is directed into the valley or concave region formed by the groove exterior to the cavity. The groove 160 may extend circumferentially around the stent graft such that the adhesive forms a ring or channel around the stent graft when it exits the holes 162. This approach may be used on either interior or exterior hollow members or folded over fabric embodiments. By directing the adhesive into a targeted location, there may be a more defined seal zone around the circumference of the stent graft and/or the hardened/cured adhesive may provide structural support for the graft material itself. As a result, a stent (e.g., metal stent ring) may not be needed in the region of the cured adhesive or a spacing between stents may be increased in the region of the cured adhesive relative to other axial regions of the stent graft.

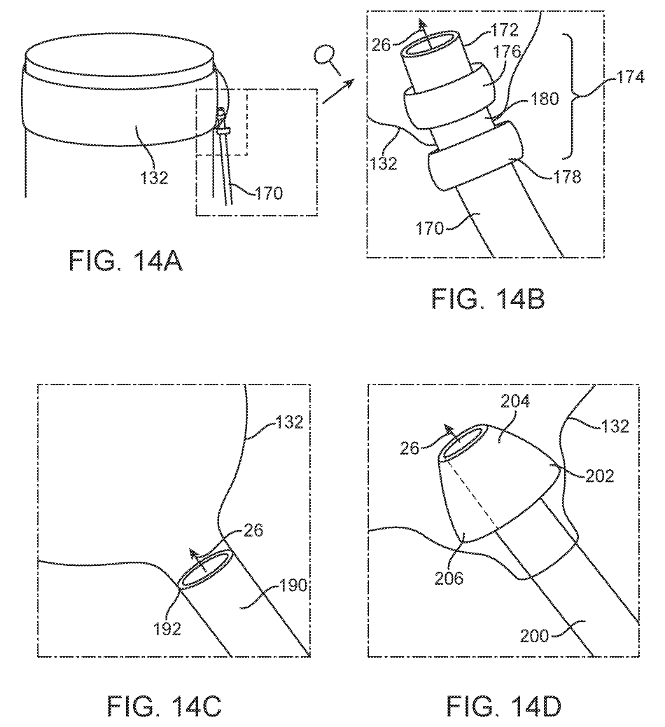

With reference to FIGS. 14A-14D, various embodiments are disclosed for fill lumens, which may be used as fill lumen 136 in the embodiments disclosed herein, for example to fill hollow member 132. FIG. 14A shows an embodiment of a fill lumen 170 being used to supply/deliver adhesive to a hollow member 132 (e.g., an external hollow member, as shown). As shown in additional detail in FIG. 14B, the fill lumen 170 may have a distal end 172 through which the adhesive 26 may be delivered through the fill lumen 170. Proximal to the distal end 172, one or more sealing members 174 may be disposed on the exterior surface of the fill lumen 170. The sealing members 174 may be configured to releasably form a seal with the hollow member 132 such that the fill lumen 170 may deliver the adhesive and subsequently be removed.

In at least one embodiment, there may be two sealing members 174 - a distal sealing member 176 and a proximal sealing member 178. The sealing members may be a gasket, such as a rubber or silicone gasket, or they may be formed of any other suitable sealing material and shape.

The distal and proximal sealing members 176, 178 may be spaced apart, such that a gap 180 is formed therebetween. The distal sealing member 176 may be configured to be disposed within the cavity 134 of the hollow member 132 and the proximal sealing member 178 may be configured to be external to the hollow member 132, with the wall of the hollow member 132 being disposed within the gap 180. Accordingly, the distal sealing member 176 may form an internal seal with an opening in the hollow member through which the fill lumen 170 is disposed, thereby preventing the adhesive from escaping the hollow member through the opening or allowing external fluids from entering the hollow member (or reducing the leakage in either direction). The proximal sealing member 178 may form an external seal with the opening in the hollow member to similarly reduce fluid flow through the opening.

In at least one embodiment, the fill lumen 170 may be inserted into the hollow member 132 prior to deployment, such that when the stent graft is expanded the fill lumen 170 is already in place and ready to deliver the adhesive. In other embodiments, the fill lumen 170 may be navigated to the opening in the hollow member 132 after the stent graft has been expanded. Once the adhesive has been delivered to the hollow member 132 through the fill lumen 170, the fill lumen 170 may be retracted from the hollow member 132 and removed from the patient. This may be accomplished by applying a downward force (in the embodiment shown) on the fill lumen 170 to cause the material of the hollow member 132 to stretch around the distal sealing member 176 and allow it to pass through.

With reference to FIG. 14C, another embodiment of a fill lumen 190 is shown. In this embodiment, sealing members may not be necessary (although could be included). Instead, the fill lumen 190 may be attached or secured to the hollow member 132, sometimes called a bag 132, by a weak bond 192 (e.g., prior to delivery). This weak bond 192 may be from an adhesive that has low strength or that weakens in response to the body environment. The adhesive used for the bond 192 may be different than the adhesive 26 delivered through the fill lumen 190. Other suitable low-strength securement mechanisms may be used, including nonadhesive mechanisms. In these embodiments, once the adhesive 26 has been delivered through the fill lumen 190, the weak bond 192 may be broken and the fill lumen 190 may be removed. The bond 192 may be broken by pulling, twisting, torqueing, or otherwise physically manipulating the fill lumen 190 to break the bond 192. If other securement mechanisms are used, suitable steps may be taken to release the fill lumen.

With reference to FIG. 14D, another embodiment of a fill lumen 200 is shown. In this embodiment, there may be a single sealing element 202 at a distal end of the fill lumen 200. The single sealing element 202 may have the form of a barb, wherein the distal end 204 has a relatively small diameter and the proximal end 206 has a relatively large diameter, with a taper therebetween. Accordingly, insertion of the fill lumen 200 into the hollow member 132 may be facilitated by the tapered shape of the sealing element 202. Similar to FIGS. 14A-B, the fill lumen 200 may be inserted into the hollow member 132 either before or after delivery of the stent graft. The larger proximal end 206 of the barb 202 may resist unintended or premature pullout of the fill lumen 200 and/or may form a seal with the hollow member 132 during filling. When the adhesive has been delivered, the fill lumen 200 may be removed by retracting it through the opening in the hollow member 132, which may stretch to accommodate the barb's passage.

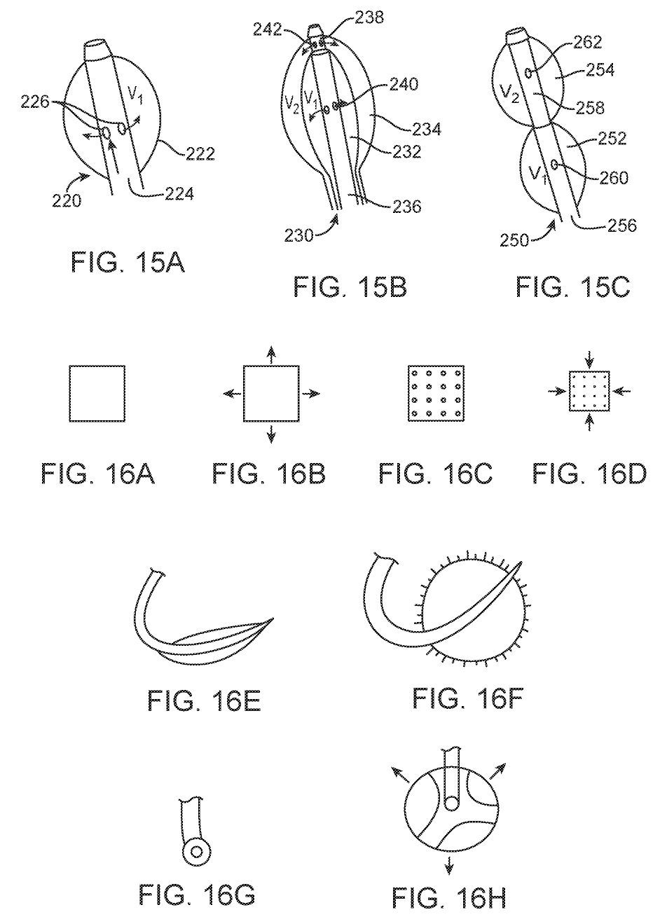

With reference to FIGS. 15A-15C, embodiments of fill balloons are described, for example, for use with other embodiments disclosed herein. With reference to FIG. 15A, a first balloon catheter 220 is shown with a balloon 222 attached to a distal end of a fill lumen 224. A fluid may be delivered to the balloon 222 through openings 226 in the fill lumen 224. As described previously and in additional detail below, adhesive may be delivered to the balloon 222 through the fill lumen 224 via the openings 226. The adhesive may be delivered through the wall of the balloon 222, such as by openings or seep holes therein.

With reference to FIG. 15B, a second balloon catheter 230 is shown with a first balloon 232 and a second balloon 234. The first balloon 232 may be disposed radially within the second balloon 234. The second balloon 234 may extend axially beyond the first balloon 232. The first balloon 232 may be attached at a distal end of a first fill lumen 236 and the second balloon 234 may be attached at a distal end of a second fill lumen 238. The second lumen 238 may extend within the first lumen 236. A fluid may be delivered to the first balloon 232 through first openings 240 in the first fill lumen 236. Another fluid may be delivered to the second balloon 234 through second openings 242 in the second fill lumen 238. The second openings 242 may be located on a portion of the second fill lumen 238 that extends distally beyond the distal end of the first fill lumen 236.

In at least one embodiment, an adhesive may be delivered to the second balloon 234 and a different fluid may be delivered to the first balloon 232. In one embodiment, the different fluid may be a liquid such as water or saline. The adhesive and the saline may be delivered simultaneously or sequentially. In one embodiment, the adhesive may be delivered first to the second balloon 234 and then the saline may be delivered to the first balloon 232. The saline may expand the first balloon 232 and exert pressure on the adhesive in the second balloon 234, thereby expelling the adhesive from the balloon (e.g., through openings or seep holes). By having nested balloons and filling one with adhesive and the other with (for example) saline, less adhesive may be used in the procedure, thereby saving costs and reducing waste of the adhesive. In one embodiment, the expanded volume V1 of the first balloon 232 is greater than the available volume V2 of the second balloon 234 (the volume not occupied by first balloon 232).

With reference to FIG. 15C, a third balloon catheter 250 is shown with a first balloon 252 and a second balloon 254. The second balloon 254 may be distal to the first balloon 252. In one embodiment, there may be no axial overlap between the two balloons 252, 254. The first balloon 252 may be attached at a distal end of a first fill lumen 256 and the second balloon 254 may be attached at a distal end of a second fill lumen 258. The second lumen 258 may extend within the first lumen 256. A fluid may be delivered to the first balloon 252 through first openings 260 in the first fill lumen 256. Another fluid may be delivered to the second balloon 254 through second openings 262 in the second fill lumen 258. The second openings 262 may be located on a portion of the second fill lumen 258 that extends distally beyond the distal end of the first fill lumen 256.

In at least one embodiment, an adhesive may be delivered to the second balloon 254 and a different fluid may be delivered to the first balloon 252. In one embodiment, the different fluid may be a liquid such as water or saline. The first balloon 252 may be inflated with, e.g., saline in order to reduce blood flow in the vessel being treated and/or to create a seal. The first balloon 252 may be inflated first and then the adhesive may be delivered through the second balloon 254 after the seal has been created or the blood flow has been reduced.

With reference to FIGS. 16A-16H, a schematic diagram of a process for creating an adhesive eluting or weeping material is shown, which may be used to form a balloon. In a first step referring to FIGS. 16A and 16B, a piece of material (FIG. 16A) may be placed in a fixture and stretched (FIG. 16B, e.g., bidirectionally). In a second step referring to FIGS. 16C and 16D, holes or openings may be created in the stretched material (FIG. 16C). When the stretch is released in the third step (FIG. 16D), the material may revert back to its original shape (e.g., elastically) and the openings may be reduced in size. In one embodiment, the openings may be effectively sealed when the stretch is released and the material returns to its original size, thereby preventing leakage therethrough. In a fourth step referring to FIG. 16E, the material may be used to form the outer wall of a balloon, such as the balloons described in the present application. Accordingly, when adhesive is delivered to the balloon, the adhesive may not initially escape through the openings due to their small size. As the balloon inflates referring to FIG. 16F, however, the material of the balloon may stretch and the openings may enlarge, thereby allowing the adhesive to flow or seep out through the openings.

With reference to FIG. 16G, in one embodiment, the balloon may or may not be generally spherical or have a circular cross-section. FIG. 16G shows an end view of one embodiment of such a balloon. In end view or in cross-section, there may be one or more channels extending in the longitudinal direction of the balloon that provide empty space in the balloon to allow blood to flow therethrough, even when the balloon is inflated. In the embodiment shown in FIG. 16H, there are three channels through the balloon, however, there may be more or less channels, such as exactly or at least 1, 2, 3, 4, 5, or more channels. The balloon may have an outer portion or perimeter that maintains contact with the vessel wall around an entire circumference of the balloon and vessel.

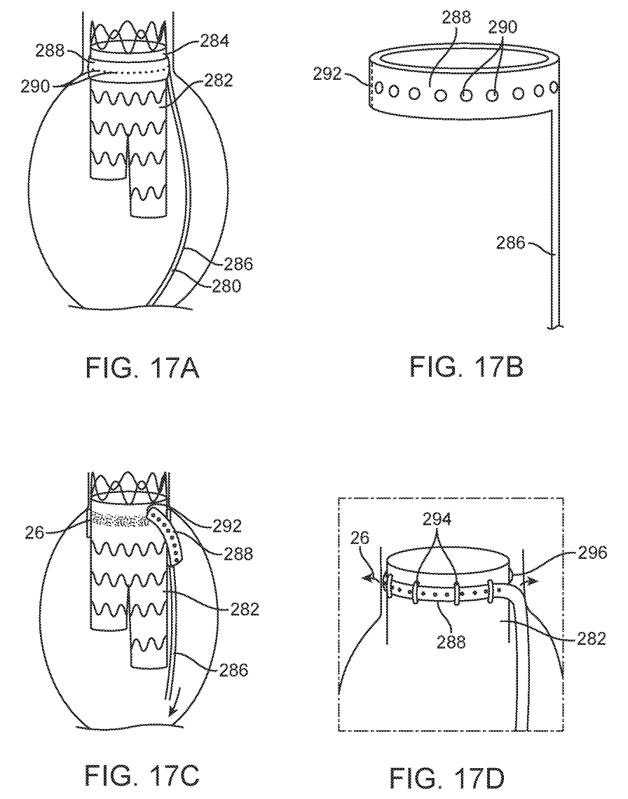

With reference to FIGS. 17A-17D, embodiments of an adhesive delivery catheter 280 are shown. The catheter 280 may be used to deliver an adhesive 26 to a circumference of a stent graft 282 or other vascular device. In the embodiment shown, the stent graft 282 may be a bifurcated stent graft, such as for a AAA procedure. The stent graft 282 may have a proximal end 284, which may form a seal with the vessel wall when the stent graft 282 is deployed. As described above, it may be beneficial to supplement the sealing force of the stent(s) of the stent graft 282 to further improve the seal of the stent graft 282 with the vessel wall and/or to reduce migration of the stent graft 282.

With reference to FIG. 17A, the catheter 280 may include a fill lumen 286, which may be configured to deliver adhesive from an adhesive source external to the patient (e.g., a syringe). The catheter 280 may further include a delivery portion 288, which may be referred to as a delivery ring 288. The delivery portion 288 may be connected to the distal end of the fill lumen 286. The delivery portion 288 may have an annular shape and may have a hollow interior which may be in fluid communication with the fill lumen 286 to receive the adhesive therefrom. The delivery portion 288 may have a plurality of openings 290 therein that may allow the adhesive to flow external to the catheter 280 from the hollow interior of the delivery portion 288. In one embodiment, the openings 290 may be spaced about the circumference of the delivery portion 288, for example, evenly spaced. The openings 290 may be on a radially external surface of the delivery portion 288 (as shown) or on a radially internal surface.

In the embodiments shown, the delivery ring 288 may be disposed about the outer surface of the proximal end 284 of the stent graft 282 during deployment of the stent graft. After the stent graft 282 has been expanded in the target location, adhesive 26 may be delivered to the delivery ring 288, where it may flow through the openings 290 to a space between the stent graft 282 and the vessel wall. Once the desired amount of adhesive 26 has been delivered, the catheter 280 may be withdrawn and the adhesive 26 may bond the stent graft 282 to the vessel wall when it cures or sets.

With reference to FIGS. 17B and 17C, an embodiment of a delivery portion 288 with a perforated region 292 is shown. The perforated region 292 may be configured to break when force is applied to the delivery portion 288, for example, a pulling force on the catheter 280 (e.g., via the fill lumen 286). The perforated region 292 may allow the delivery ring 288 to split, thereby allowing it to disengage from around the tubular stent graft (FIG. 17C ) and be removed from the patient. In one embodiment, the perforated region 292 may be located on the delivery portion 288 opposite the connection to the fill lumen 286, for example, diametrically opposed. This may allow the delivery ring 288 to split into two equal sized portions on opposite sides of the fill lumen 286 and may facilitate a balanced splitting force being applied to the perforation. While a perforation 292 is shown, any mechanism for allowing the delivery portion to split the delivery portion 288, such as a weakened or thinner region of material, a releasable fastener (e.g., hook and loop), a releasable knot, etc. may be used.

With reference to FIG. 17D, an embodiment is shown of a delivery portion 288 being temporarily secured to the stent graft 282 by one or more filaments 294. Filaments 294 may be any type of thin, elongated material, such as sutures, wires, yarn, etc. The filament(s) 294 may secure the delivery portion 288 to the stent graft 282, either to the graft material, the stents, or both. The filament(s) 294 may form a loop around the delivery portion 288 to prevent it from sliding axially up or down on the stent graft 282. In this embodiment, instead of splitting a perforation, the delivery portion 288 may not be formed in a complete ring, but may be a normally straight tube that is bent or curled around the stent graft 282. As shown in FIG. 17D, in this embodiment the distal end 296 of the delivery portion 288 is a free end and does not connect to the rest of the delivery portion (e.g., in a ring). To remove the adhesive delivery catheter 280, it may be pulled or retracted such that the delivery portion 288 slides out from the loops and disengages from the stent graft 282. In one embodiment, the filaments 294 may be released to allow the delivery portion 288 to be removed.

While the delivery catheter 280 is shown for the purposes of sealing a proximal end of a stent graft to a vessel wall, it may also be used to seal the distal end. There may also be multiple delivery catheters 280 configured to deliver adhesive to multiple locations, such as one at the proximal end and one at the distal end. For a stent graft assembly, such as a AAA assembly with a proximal seal zone and two seal zones in the iliac arteries, each seal zone may have a delivery catheter 280 associated therewith to improve the seal. In addition to, or alternatively, using the delivery catheter 280 to seal an external surface of a stent graft to a vessel wall, the delivery catheter may be used to improve the connection between two stent grafts. For example, the delivery portion 288 may be disposed about the external surface of a proximal end of a stent graft that is to be deployed within the distal end of a previously deployed stent graft. The adhesive may then be delivered to improve the bond and seal between the two stent grafts. Alternatively, the delivery portion may be disposed on the inner circumferential surface of the first deployed stent graft.

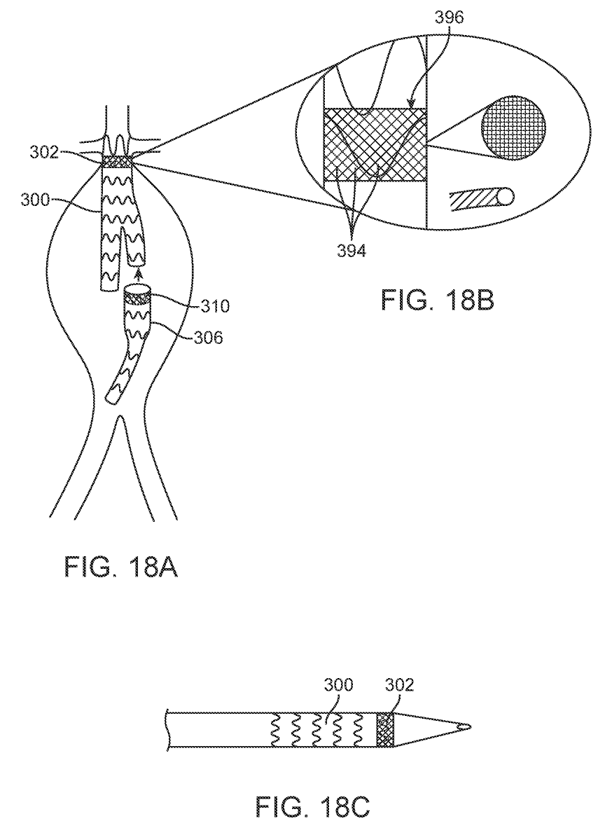

With reference to FIGS. 18A-18C, embodiments are shown of stent grafts with an adhesive material incorporated therein prior to deployment. Embodiments have been described herein of delivering adhesive to a stent graft, e.g., using a delivery catheter, during or after deployment of the stent graft. However, in some embodiments, the adhesive may be incorporated into the stent graft prior to deployment. With reference to FIG. 18A, a first stent graft 300 is shown, in this example a AAA bifurcated stent graft. The first stent graft 300 may be similar to other stent grafts described herein having a graft material and one or more stents attached thereto. The first stent graft 300 may have a proximal seal zone 302, which may be configured to seal the stent graft at the proximal end of an aneurysm. In at least one embodiment, the graft material of the proximal seal zone 302 may have an adhesive material incorporated therein, e.g., directly or integrally incorporated therein. The adhesive in the proximal seal zone 302 may cause the stent graft 300 to bond or adhere to the vessel wall, thereby improving the seal of the stent graft and reducing/preventing migration. The adhesive may be disposed primarily only on the outer surface of the proximal seal zone 302 that will contact the vessel wall or it may be disposed throughout the thickness of the graft material. While a proximal seal zone is shown and described, a distal or other seal zone may also or alternatively include the adhesive.

The adhesive in the proximal seal zone 302 may be incorporated into the graft material around a full circumference of the graft material. In the example shown, the adhesive may be present in a cylindrical section of the graft - covering the full circumference for a defined height. However, the adhesive may also be formed as a single line or narrow strip around the circumference and may not have a significant height. In addition, the adhesive may not be formed around the entire circumference, but may be discontinuous or have spaced apart sections with no adhesive in between (e.g., such as a dashed line).

With reference to FIG. 18B, there may be various ways to incorporate the adhesive into the graft material. In one embodiment, there may be at least a subset (e.g., some or all) of yarns 394 within the weave 396 of the graft material that have adhesive incorporated therein. The yarns 394 may be formed of the adhesive material itself (top right) or the yarns may be coated with the adhesive material (bottom right). In another embodiment, the graft material may be impregnated with the adhesive, for example, by pressing, dipping, spraying, submerging, painting, or otherwise applying the adhesive to the graft material such that it penetrates the pores of the material and is embedded therein. The adhesive may also be applied as a coating that rests substantially on top of the material, at least until a pressure is applied to cause it to penetrate (e.g., a balloon expansion).

The adhesive in these embodiments may be configured to cure or set relatively slowly such that it does not adhere or fully adhere until it is in contact with the vessel. Accordingly, the adhesive may still allow the stent graft to be compressed and loaded into a delivery configuration, such as shown in FIG. 18C, without curing. This may be accomplished by having a slow cure rate or the adhesive may be activated by contact with blood, such that it does not begin to cure until it is inside the body. In another embodiment, the adhesive may be covered by a protective sheet or membrane prior to deployment, which may then be removed either prior to expansion or after expansion of the stent graft. In yet another embodiment, the protective sheet or membrane may be dissolvable or resorbable such that it deteriorates within the body and exposes the adhesive after a certain amount of time. The adhesive need not cure immediately upon stent graft expansion. In another embodiment, the adhesive may be disposed within a protective membrane or pouch such that it is not exposed to the body. The membrane or pouch may be selectively rupturable when exposed to a threshold level of pressure in order to release the adhesive in the desired area of the membrane/pouch. For example, a balloon may be inflated within the stent graft such that it contacts the membrane or pouch and exceeds the rupture strength thereof to cause it to burst and release the adhesive. The adhesive may then perform similarly to above, creating a bond between the stent graft and another stent graft or a vessel wall.

In addition to, or alternatively, to improving a seal zone with the vessel wall, the incorporated adhesive may be used to improve the joint strength of two stent grafts. A second stent graft 306 is shown in FIG. 18A, with a proximal joint zone 310, which may be generally similar to the proximal seal zone 302 described above. When the second stent graft 306 is deployed within a portion of first stent graft 300 (e.g., the distal end of a bifurcated limb, as shown), the adhesive in the proximal joint zone 310 may adhere or bond to the graft material and/or stents in the first stent graft 300 to secure the two stent grafts together, in addition to the mechanical/interference bond formed by overlapping and radial force. While the joint zone is shown on the outer surface of the proximal end of the second stent graft 306, it may alternatively be formed on an inner surface of the distal portion of the first stent graft 300 to have a similar effect (or the adhesive may be throughout the thickness of the graft material). In other embodiments, both joint zones may have adhesive incorporated therein. While certain example joint zones have been described, it should be understood that the graft material with incorporated adhesive may be used in any portion of a stent graft (or other endovascular device) that is desired to be secured to a vessel or to another device.

Any suitable biocompatible adhesive or bioglue may be used as the adhesive in the disclosed embodiments. In one embodiment, the adhesive may be activated or polymerized by contact with blood. One example of an adhesive may be a cyanoacrylate based adhesive, e.g., 2-octyl cyanoacrylate, and/or a sclerosing agent such as hypertonic saline, sodium tetradecyl sulfate, chromated glycerol, tetracycline, talc, bleomycin, or polydocanol. In some embodiments, a cyanoacrylate can be an aliphatic 2-cyanoacrylate ester such as an alkyl, cycloalkyl, alkenyl or alkoxyalkyl 2-cyanoacrylate ester. The alkyl group may have from 1 to 16 carbon atoms in some embodiments, and can be a C1-C8 alkyl ester or a C1-C4 alkyl ester. Some possible esters include the methyl, ethyl, n-propyl, isopropyl, n-butyl, isobutyl, pentyl, hexyl, cyclohexyl, heptyl, octyl, 2-methoxyethyl and 2-ethoxyethyl esters of cyanoacrylic acid. Other adhesives that can be used include a biological glue such as a bovine serum albumingluteraldehyde combination (e.g., BIOGLUE, Cryolife, Atlanta, Ga.), PVA, Biogard, collagen, fibrinogen, fibronectin, vitronectin, laminin, thrombin, gelatin, mixtures thereof, or other biocompatible adhesives. Further examples of adhesives and their properties (e.g., curing times, additives, etc.) may be found in U.S. Patent Numbers 8,475,492 and 8,845,614 the entireties of which are hereby incorporated by reference herein.

While various embodiments have been described herein for using adhesives with stent grafts, any embodiments described herein may also be applicable to other endovascular devices. For example, the adhesive systems and methods may be applied to other devices, such as artificial valves (e.g., heart valves), bare stents, covered stents, or others.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

While exemplary embodiments are described above, it is not intended that these embodiments describe all possible forms. The words used in the specification are words of description rather than limitation, and it is understood that various changes can be made without departing from the spirit and scope of the disclosure. As previously described, the features of various embodiments can be combined to form further embodiments of the invention that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. These attributes can include, but are not limited to cost, strength, durability, life cycle cost, marketability, appearance, packaging, size, serviceability, weight, manufacturability, ease of assembly, etc. As such, to the extent any embodiments are described as less desirable than other embodiments or prior art implementations with respect to one or more characteristics, these embodiments are not outside the scope of the disclosure and can be desirable for particular applications.

What is claimed is:

1. A method comprising:
navigating a primary stent graft to a landing zone of a vessel;
expanding the primary stent graft;
navigating an adhesive catheter within the primary stent graft;
deploying a secondary stent graft within the primary stent graft such that the adhesive catheter is trapped between the primary and secondary stent grafts;
dispensing an adhesive from the adhesive catheter; and
bonding the primary stent graft to the secondary stent graft with the adhesive.

2. The method of claim 1, wherein the expanding the primary stent graft includes expanding the primary stent graft to contact the landing zone.

3. The method of claim 2, wherein the deploying is subsequent to the expanding.

4. The method of claim 2 wherein the dispensing is subsequent to the expanding and the deploying.

5. The method of claim 1, wherein the landing zone is within an aorta between renal arteries and an abdominal aneurysm.

6. The method of claim 1, wherein the adhesive prevents migration of the secondary stent graft relative to the primary stent graft.

7. The method of claim 1 wherein the dispensing occurs from the adhesive catheter between the primary stent graft and the secondary stent graft deployed within the primary stent graft.

8. The method of claim 1, wherein the dispensing the adhesive comprises dispensing the adhesive within a circumferential region of application.

9. The method of claim 1, further comprising applying outward pressure on the secondary stent graft.

10. A method comprising:
navigating a primary stent graft to a landing zone of a vessel;
expanding the primary stent graft;
navigating an adhesive catheter within the primary stent graft and positioned radially within it;
deploying a secondary stent graft within the primary stent graft;
dispensing an adhesive from the adhesive catheter; and
bonding the primary stent graft to the secondary stent graft with the adhesive.

11. The method of claim 10, wherein the adhesive catheter contacts an inner surface of the primary stent graft after the navigating.

12. The method of claim 10, wherein the adhesive catheter contacts an outer surface of the secondary stent graft after the deploying.

13. The method of claim 10, wherein the deploying is subsequent to the expanding.

14. The method of claim 10, wherein the dispensing is subsequent to the expanding and the deploying.

15. The method of claim 10, wherein the dispensing the adhesive comprises dispensing the adhesive within a circumferential region of application.

16. A method comprising:
navigating a primary stent graft to a landing zone of a vessel;
expanding the primary stent graft;
navigating an adhesive catheter within the primary stent graft;
partially deploying a secondary stent graft within the primary stent graft;
dispensing an adhesive from the adhesive catheter while the secondary stent graft is in its partially deployed state; and
bonding the primary stent graft to the secondary stent graft with the adhesive.

17. The method of claim 16, further comprising fully deploying the secondary stent graft.

18. The method of claim 16, further comprising inflating a balloon in a region where the primary and secondary stent grafts overlap and adhesive has been dispensed to force the first and secondary stent grafts together.

19. The method of claim 16, further comprising removing the adhesive catheter after the dispensing.

20. The method of claim 16, wherein a portion of the secondary stent graft is in a compressed state after the partially deploying.
